(19) United States
(12) Patent Application Publication
(10) Pub. No.: US 2020/0375749 A1
(43) Pub. Date: Dec. 3, 2020

(54) One-Stage CAD/CAM Facial Skeletal Rearrangement and Refinement

(71) Applicant: Michael J. Yaremchuk, Lynnfield, MA (US)

(72) Inventor: Michael J. Yaremchuk, Lynnfield, MA (US)

(21) Appl. No.: 16/891,710

(22) Filed: Jun. 3, 2020

Related U.S. Application Data
(60) Provisional application No. 62/856,554, filed on Jun. 3, 2019.

(51) Int. Cl. A61F 2/30; A61F 2/28; A61B 34/10; G16H 50/50; G16H 50/20; G16H 30/40; G16H 20/40; G06F 30/10

(52) U.S. Cl. CPC A61F 2/30942; A61F 2/2875; A61B 34/10; G16H 50/50; A61F 2002/4633; G16H 30/40; G16H 20/40; G06F 30/10; G16H 50/20

(57) ABSTRACT

A method for corrective surgery with a rearrangement of skeletal features, such as facial skeletal features, of a patient and a refinement of irregularities by alloplastic implant in a single surgery. The method includes determining whether the patient is a candidate for corrective surgery and pre-operatively imaging skeletal features to be rearranged to produce three-dimensional computer renderings of the skeletal features. The rearrangement of skeletal features is pre-operatively planned, and skeletal irregularities to remain after the rearrangement of skeletal features are pre-operatively predicted. Based on the skeletal irregularity predicted to remain after the rearrangement of skeletal features, an alloplastic implant is pre-operatively designed and manufactured by computer-aided design and manufacture. In a single surgery, skeletal features are rearranged in accordance with the pre-operative planning, and the alloplastic implant is applied to the skeletal features in correction of the predicted skeletal irregularity.

### RELATED APPLICATION

This application claims priority to U.S. Provisional Patent Application No. 62/856,554, filed Jun. 3, 2019, which is incorporated herein by reference.

### FIELD OF THE INVENTION

The present invention relates generally to corrective surgery. More particularly, disclosed herein is a method for corrective surgery wherein the rearrangement of skeletal features and the refinement of resulting contour irregularities and facial imbalances are carried out in a single operative procedure through computer-aided design (CAD) and computer-aided manufacture (CAM).

### BACKGROUND OF THE INVENTION

Facial skeletal surgery has been revolutionized in recent years by advances not only in biomaterials but also in computerized tomographic (CT) imaging. The multiple images provided by CT imaging of skeletal characteristics has markedly increased the amount of available data compared to that previously provided by plane radiographs. Moreover, the incorporation of three-dimensional computerized tomographic (3D CT) imaging, including by the present inventor, has enabled the determination of the shape and volume of anatomic structures in both normal and abnormal conditions. Reference will be had, for example, to the publication “The role of 3D CT in the evaluation of acute craniofacial trauma” in Ann Plast Surg 1993; 31:488-494 by Broumand S R, Labs J D, Novelline R A, Markowitz B L, Yaremchuk M J. Furthermore, miniaturized plates and screws formed of biocompatible metals, such as titanium, have been developed to stabilize repositioned segments of the facial skeleton, again including by the present inventor as described in “Rigid fixation of the craniomaxillofacial skeleton” by Yaremchuk M J and Gruss J S, Manson P N, editors, Boston: Butterworth-Heinemann, 1992.

Computers and computer software have thus been used not only to design biocompatible implants in a process referred to as computer-aided design (CAD) but also to control manufacturing in a process commonly referred to as computer-aided manufacture (CAM). When used in combination, computer-aided design (CAD) and computer-aided manufacture (CAM) permit the efficient design and production of components, including biocompatible implants, in a combined CAD/CAM process. CAD/CAM technology is currently used to implement facial skeletal rearrangement operative procedures and in secondary procedures to refine contour irregularities resulting from the original skeletal rearrangement procedures.

Employing such systems and methods, midface and mandible orthognathic surgery can be performed, such as with titanium plating fixed in place by titanium screws. Also, as described by the present inventor in the “Atlas of Facial Implants,” Philadelphia: Saunders-Elsevier, 2007, Yaremchuk, M J, biocompatible plastics, such as methacrylates and polyethylene, have been designed to replace missing skull segments and to augment skeletal contours. For instance, methyl methacrylate has been positioned over titanium mesh to reconstruct bone skull defects.

It is also known to use alloplastic biomaterials in CAD/CAM processes to reconstruct skull defects. For instance, with U.S. Patent Application Publication No. 2006/0094951, which later issued as U.S. Pat. No. 7,747,305, Dean et al. teach the Computer-aided-design of Skeletal Implants wherein an implant for a patient is designed, validated, and fabricated based on data acquired through three-dimensional scanning of the patient's defect site. With U.S. Patent Application Publication No. 2009/149977 for Methods, Systems, and Computer Program Products for Shaping Medical Implants Directly from Virtual Reality Models, Schendell references the potential for CAD/CAM technology to advance facial skeletal surgery, writing, “Using the graphics software, the virtual model of the patient's skull can be cut and manipulated into a desired configuration as one would in the actual surgery.”

CAD/CAM technology is thus now routinely used in elective facial skeletal surgery, such as orthognathic and free fibula mandible reconstruction. For instance, U.S. Patent Application Publication No. 2017/0000564 of Gordon is directed to a Computer-Assisted Planning and Execution System wherein orthognathic and other surgeries are performed with surgical cutting planes determined on a computer-readable representation of a donor skeletal fragment. The donor skeletal fragment is positioned within a transplant region of a recipient skeletal fragment, and a hybrid computer-readable representation is created comprising the recipient skeletal fragment and the portion of the donor skeletal fragment.

Further, with U.S. Pat. No. 10,595,942, Rueber et al. disclose visualizing a bone model on a display device, deriving plate design data, and generating a data set that geometrically defines a bone plate design. Through multiple surgical procedures, a mandible can thus be reconstructed by replacing a removed bone portion with bone material taken, for example, from a fibula or rib that itself is replaced in a later surgical procedure. The results of such a mandible reconstruction procedure employing CAD are depicted in FIGS. 2A and 2B. There, in a skeletal rearrangement procedure, segments of the lower maxilla 102 were advanced, the body of the mandible 104 was recessed, and the chin 106 was advanced through the planning and execution of an orthognathic surgery procedure. Meanwhile, FIG. 3 illustrates the result of an orthognathic procedure with CAD/CAM involving movements of the maxilla 102 and the mandible 104, including by the use of plates 105 and screws 107 to maintain fixation of the designed skeletal movement.

A practice of skeletal rearrangement wherein donor bone sections 110 and 112 of a fibula bone 108 are used in the construction of a section of a mandible 104 can be further understood with reference to FIGS. 4A through 4D. As depicted, a portion of a patient's fibula bone 108 was osteomized, repositioned, and stabilized using CAD/CAM technology. In FIGS. 4A and 4B, one can perceive planning as a result of a 3D CT image of the patient's fibula bone 108. Locations of desired bone cuts in the fibula 108 are identified with cutting guides to allow the creation of bone segments 110 and 112 and the rearrangement of those bone segments 110 and 112 to create the desired contour of the mandible 104 as depicted in FIGS. 4C and 4D. Appropriate location and placement of the osteotomy is obtained through an intraoperative view of CAD/CAM cutting guides. As shown in the postoperative images of FIGS. 4C and 4D, the repositioned fibula bone segments 110 and 112 are fixed in place in desired locations and orientations by plate and screw fixation. As depicted, the repositioned fibula bone segments 110 and 112 are fixed in place to reconstruct and approximate the overall shape of the mandible 104.

Unfortunately, while the overall shape of the mandible 104 may be recreated as in FIGS. 4C and 4D, significant skeletal contour irregularities and imbalances typically remain after facial skeletal rearrangement procedures. Such irregularities and imbalances can be seen, for instance, in FIGS. 2A, 2B, 4C, and 4D. Contour irregularities, imbalances, and deficiencies commonly remain externally apparent after a skeletal rearrangement intended to achieve a given contour has been completed. Because a deficient structure has been rearranged, the structure remains deficient but now in a different way. The result is a structure with contour irregularities, imbalances, and deficiencies.

The present inventor has used CAD/CAM to create biomaterial implants to correct irregularities resulting from such skeletal rearrangement surgeries as is described, for instance, in “Refining Post-Orthognathic Surgery Facial Contour with Computer-Designed/Computer-Manufactured Alloplastic Implants,” Plastic and Reconstructive Surgery: September 2018—Volume 142 by Lee, Jeffrey H., M.D.; Kaban, Leonard B., M.D., D. M. D.; Yaremchuk, Michael J., M.D. and in “Atlas of Facial Implants Second edition,” Philadelphia: Saunders-Elsevier, 2020, by Yaremchuk M J, Chang, C S, Dayan E, Mrad M A, Yan A.

The correction of irregularities using alloplastic implants can be further understood with reference to FIGS. 5A and 5B. A skeleton after orthognathic surgery on the maxilla 102 and the mandible 104 is depicted in FIG. 5A. There, one can perceive resultant contour irregularities, such as those indicated at 114 and 116, after the first skeletal rearrangement orthognathic surgery. With the contour irregularities 114 and 116 apparent after the first surgery has been completed, a second, post-orthognathic refinement surgery must be carried out to refine and otherwise rectify the results of the first, skeletal rearrangement surgery. For instance, as depicted in FIG. 5B, with the contour irregularities 114 and 116 of FIG. 5A apparent after the skeletal rearrangement surgery, alloplastic implants 118, 120, and 122 were created through CAD/CAM practices, and a later refinement surgery was performed during which the CAD/CAM created implants 118, 120, and 122 were placed to overlie and otherwise to refine and rectify the contour irregularities 114 and 116. Through the subsequent refinement surgery, the skeletal contours were thus improved as the contoured surfaces of the implants 118, 120, and 122 were installed to overlie the contour irregularities 114 and 116 and to create a desired refined skeletal contour.

The ability to refine and correct contour irregularities through refinement surgery to install alloplastic implants is certainly advantageous for the results achieved. However, the need for one or more subsequent refinement surgical procedures to be planned, performed, and recovered from post-operatively after there has already been a skeletal rearrangement surgery with its own planning, performance, and post-operative recovery increases the trauma to the patient, elevates patient morbidity, and increases surgical time, convalescence, and expense.

With a deep knowledge of the foregoing, the present inventor has appreciated that it would represent a marked advance in the art of corrective surgery if the rearrangement of facial skeletal features and the refinement of resulting contour irregularities and imbalances could be achieved during a single operative procedure.

### SUMMARY OF THE INVENTION

The present invention is thus founded on the basic object of enabling corrective surgery involving the rearrangement of skeletal features and the refinement of resulting contour irregularities and facial imbalances to be carried out in a single operative procedure.

In practices of the invention, facial skeletal rearrangement and the refinement of irregularities and imbalances are done during a single operative procedure with the use of computer-aided design (CAD) and computer-aided manufacture (CAM).

An underlying object of the invention is to minimize or eliminate the need for secondary refinement surgeries after skeletal rearrangement and the trauma, planning, increased morbidity, and expense involved therein.

An further object of the invention is to enable facial skeletal rearrangement procedures to be carried out with improved refinement and balance through a single operative effort.

These and in all likelihood further objects and advantages of the present invention will become obvious not only to one who reviews the present specification and drawings but also to those who experience the one-stage CAD/CAM facial skeletal rearrangement and refinement disclosed herein in practice. Although the accomplishment of each of the foregoing objects in a single embodiment of the invention may be possible and indeed preferred, not all embodiments will seek or need to accomplish each and every potential advantage and function. Nonetheless, all such embodiments should be considered within the scope of the present invention.

In one embodiment of the invention, a method for corrective surgery is provided with a rearrangement of skeletal features, such as facial skeletal features, of a patient and a refinement of irregularities by alloplastic implant in a single surgery. The method can include determining whether the patient is a candidate for corrective surgery, which can include evaluation of the skeletal features to be rearranged by three-dimensional computerized tomographic imaging. Based on pre-operative imaging, three-dimensional computer renderings of the skeletal features are produced. Computer-aided design is employed to perform pre-operative planning of the rearrangement of skeletal features. Computer-aided design is also exploited to predict pre-operatively one or more skeletal irregularities that will remain after the rearrangement of the skeletal features and to pre-operatively design an alloplastic implant based on the predicted skeletal irregularity.

The alloplastic implant can be pre-operatively manufactured based on the computer-aided design, such as through an additive manufacturing process. Furthermore, a three-dimensional computer file of the alloplastic implant can be created, and the alloplastic implant can be manufactured through computer-aided manufacture based on the computer-aided design and the three-dimensional computer file.

In accordance with the pre-operative planning of the rearrangement of skeletal features, the skeletal features are rearranged, and the alloplastic implant is applied to the skeletal features in correction of the predicted skeletal irregularity. Both the rearrangement of skeletal features and the application of the alloplastic implant to the skeletal features are performed in a single surgery thereby improving immediate results and reducing patient trauma and the planning, expense, and increased morbidity as compared to requiring a secondary refinement surgery in addition to an original skeletal rearrangement surgery.

In practices of the invention, pre-operative imaging and the rearranging of skeletal features can be performed under computerized tomographic imaging guidance. Furthermore, the step of pre-operatively planning the rearrangement of skeletal features with computer-aided design can include planning by computer of osteotomies and the step of designing cutting guides for the osteotomies by computer-aided design.

One will appreciate that the foregoing discussion broadly outlines the more important goals and features of the invention to enable a better understanding of the detailed description that follows and to instill a better appreciation of the inventor's contribution to the art. Before any particular embodiment or aspect thereof is explained in detail, it must be made clear that the following details of construction and illustrations of inventive concepts are mere examples of the many possible manifestations of the invention.

### BRIEF DESCRIPTION OF THE DRAWINGS

In the accompanying drawing figures:

FIG. 1 is a schematic view of a process for facial skeletal rearrangement and refinement through a single stage of surgical operation according to the invention;

FIGS. 2A and 2B are views in front and side elevation of a skeleton after a mandible reconstruction procedure;

FIG. 3 is a view in front elevation of a skeleton after an orthognathic procedure involving movements of the maxilla and the mandible;

FIGS. 4A and 4B are plan views of a fibula bone prepared for osteotomy for the creation of bone segments for mandible reconstruction;

FIGS. 4C and 4D are views in front and side elevation of a mandible reconstructed with fibula bone segments;

FIG. 5A is a view in side elevation of a skeleton after orthognathic surgery on the maxilla and the mandible with resultant contour irregularities; and

FIG. 5B is a view in side elevation of the skeleton of FIG. 5A with the contour irregularities refined by alloplastic implants.

### DETAILED DESCRIPTION OF PREFERRED EMBODIMENTS

The methods for one-stage facial skeletal rearrangement and refinement using computer-aided design and computer-aided manufacture disclosed herein are subject to a wide variety of embodiments, each within the scope of the invention. However, to ensure that one skilled in the art will be able to understand and, in appropriate cases, practice the present invention, certain preferred embodiments of the broader invention revealed herein are described below and shown in the accompanying drawing figures.

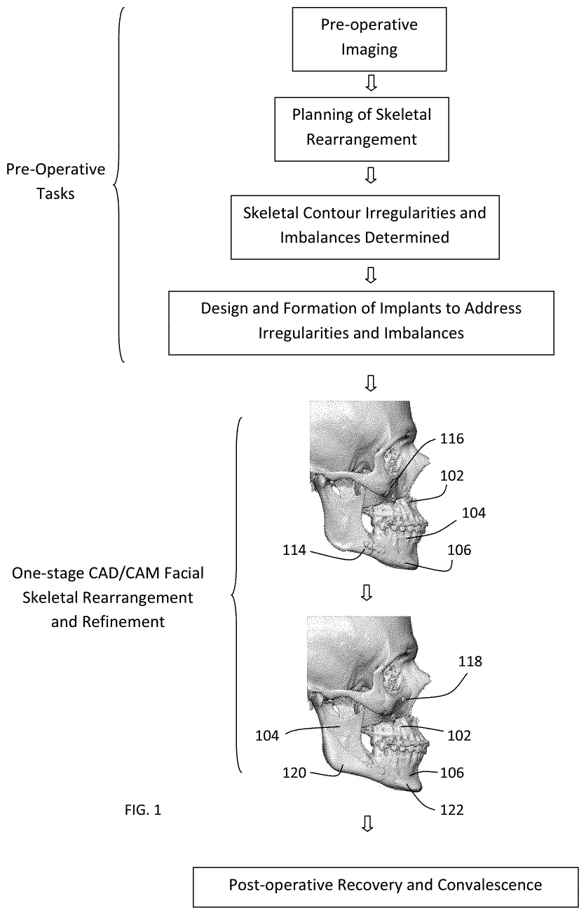

The present invention overcomes the challenges of the prior art with respect to the need for subsequent refinement surgery by a method for corrective surgery wherein skeletal facial features are rearranged and the contour irregularities and imbalances that would otherwise derive therefrom are refined and corrected with one or more implants in a single operative procedure. A non-limiting but illustrative practice of the present invention for facial skeletal rearrangement and refinement with a single-stage surgical procedure is depicted schematically in FIG. 1. There, the inventive method is practiced in relation to a facial skeletal rearrangement wherein orthognathic surgery is performed on the maxilla 102 and the mandible 104 leaving contour irregularities and imbalances, such as those indicated at 114 and 116.

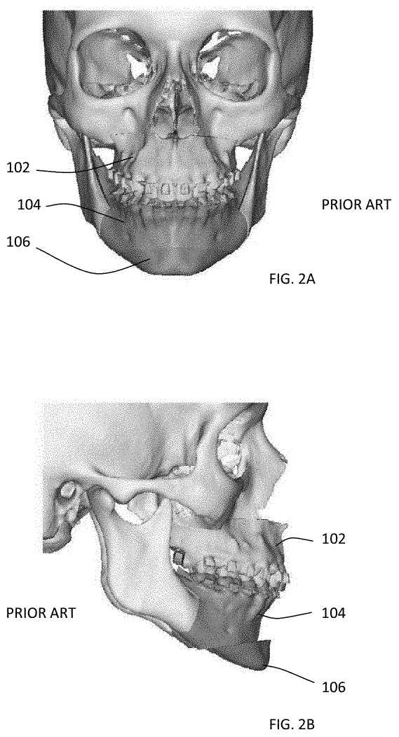

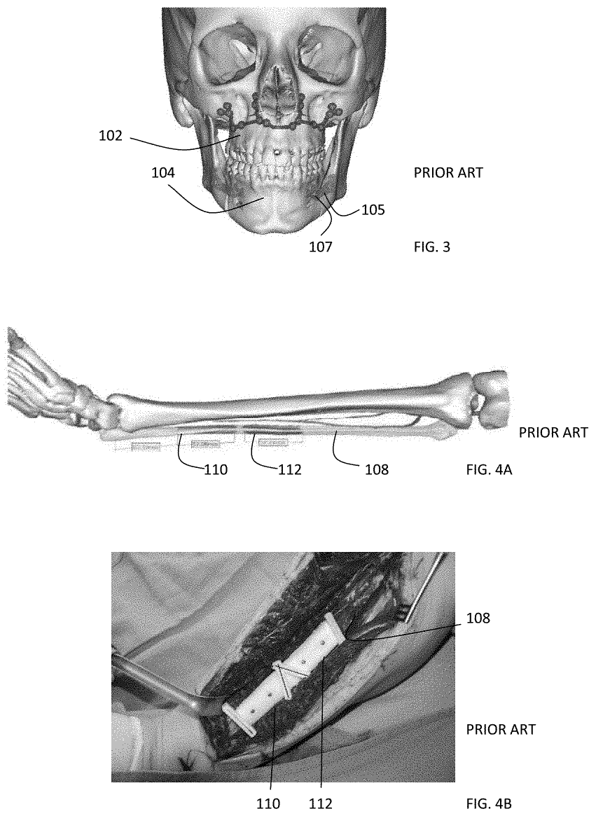

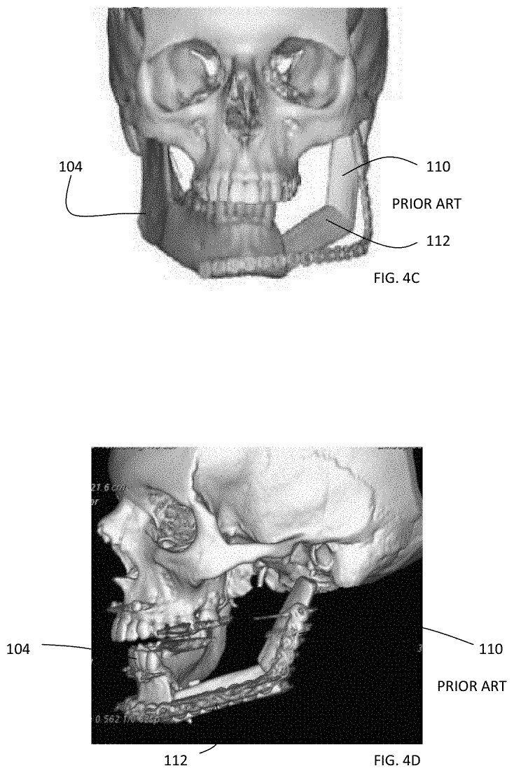

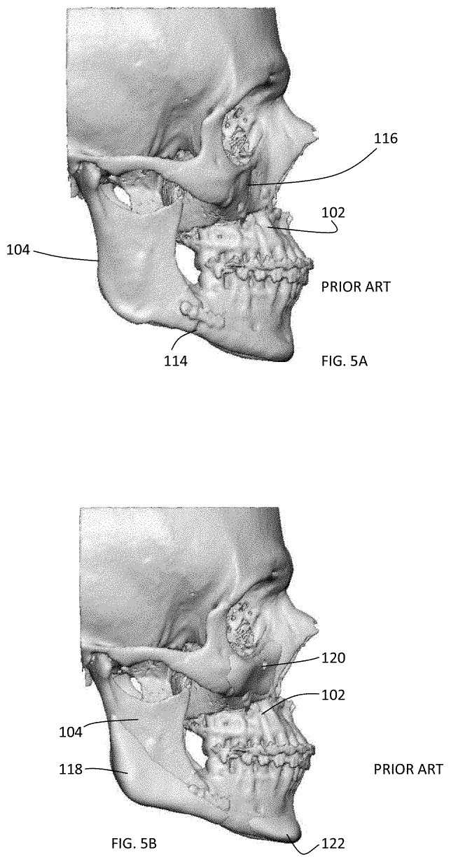

It will be understood that the particular type of skeletal rearrangement procedure can vary within the scope of the invention except as it might be expressly limited by the claims. Other non-limiting skeletal rearrangement procedures could, for example, comprise the rearrangement of one or more of the lower maxilla 102, the body of the mandible 104, and the chin 106 as in FIGS. 2A, 2B, and 3 or fibula mandible reconstruction of the mandible 104 through the fixation of one or more bone sections 110 and 112 as in FIGS. 4A through 4D. As used herein, facial skeletal rearrangement shall be interpreted to include but not be limited to skeletal repair, reconstruction, reconfiguration, osteotomy, and any other skeletal rearrangement where a contour irregularity or imbalance may result. Also, as used herein, contour irregularities and imbalances may be generally referred to as irregularities.

In the depicted practice of the one-stage facial skeletal rearrangement and refinement procedure using CAD/CAM, the procedure begins with a determination as to whether the patient would be an appropriate candidate for CAD/CAM facial skeletal rearrangement and refinement. That determination can include pre-operative imaging. The pre-operative imaging can, for instance, be radiologic imaging, potentially three-dimensional computerized tomographic (3D CT) imaging of the skeletal area under consideration. The 3D CT imaging produces one or more three-dimensional computer renderings of the skeletal area.

Based on the pre-operative imaging, such as, but not limited to, 3D CT imaging, the surgical team, in cooperation with the patient as appropriate, can undertake pre-operative planning of the skeletal rearrangement to be performed. The subject skeletal area and the skeletal rearrangement can be simulated virtually through three-dimensional computer analysis and display. The planning can further include the computerized design and creation through CAD/CAM of unique skeletal cutting guides and fixation plates based on the 3D CT imaging of the skeletal area and the planned skeletal rearrangement.

Through electronic modeling provided by three-dimensional CAD/CAM computer software, three-dimensional electronic data is produced pre-surgically virtually depicting and evidencing on a computer display any contour irregularities and imbalances that would result from the planned skeletal rearrangement. Based on the predicted contour irregularities and imbalances, one or more alloplastic implants can then be pre-surgically designed in three dimensions and dependent on the electronically predicted irregularities and imbalances to address those irregularities and imbalances using computer software to produce a three-dimensional computer rendering of the implant or implants and a three-dimensional computer file retaining that rendering in electronic memory. The three-dimensional computer file can then be employed, such as through three-dimensional printing or any other formation technique, to create actual implants uniquely designed and formed based on the predicted contour irregularity or imbalance to address the contour irregularity or imbalance pre-operatively before the skeletal rearrangement that will ultimately produce the irregularity or imbalance is initiated.

The invention is not in any way limited as to the material of the implants or as to the technique for forming the same. As used herein, reference to alloplastic material shall be interpreted to include any biocompatible material suitable for a surgical implant. Possible, but not limiting, materials include silicone rubber, polyethylene, polyethyletherketone, methacrylate, and any other biocompatible material. Furthermore, while additive manufacturing may be advantageously used within the scope of the invention, any other effective technique could be used within the scope of the invention except as it might be expressly limited by the claims. Again without limitation, implants could alternatively be formed by molding of biocompatible material or by any other method.

Accordingly, prior to the start of a one-stage skeletal rearrangement and refinement surgery pursuant to the present invention, the surgical team is in possession not only of planning for the skeletal rearrangement, unique skeletal cutting guides, and any needed fixation plates and fasteners but also of each alloplastic implant deemed necessary to address the contour irregularities and imbalances that are predicted to derive from the yet-to-be-performed skeletal rearrangement. Equipped with the information and tools required to perform the skeletal rearrangement precisely and to address the contour irregularities and imbalances that will derive therefrom, the surgeon can perform the skeletal rearrangement, whether it be fibula mandible reconstruction, orthognathic surgery, or another facial skeletal rearrangement. Then, in the same surgery and prior to surgical closure or post-operative recovery and convalescence from the skeletal rearrangement, the surgeon can install the alloplastic implants as in the non-limiting depiction of FIG. 1 to address the irregularities and imbalances 114 and 116 that had been predicted before the one-stage surgical procedure had begun.

With the skeletal rearrangement completed and the resulting irregularities and imbalances addressed by the application of implants uniquely and specifically designed and formed pre-surgery based on three-dimensional CAD/CAM imaging and planning electronic data, the one-stage surgical procedure can be completed. The patient is provided with the planned skeletal rearrangement and the rectification of irregularities and imbalances deriving from the rearrangement in a single, one-stage skeletal rearrangement and refinement surgery. Rather than is the case with plural separate surgeries for skeletal rearrangement and then later refinement of irregularities and imbalances, a single 3D CT pre-operative imaging session can be followed by a single planning session, a single operation, and a single post-operative recovery and convalescence.

According to the invention, therefore, CAD/CAM can be employed during the design process to achieve a CAD/CAM orthognathic procedure. By predicting what the irregularities 114 and 116 will be before the surgery, one practicing the present invention can immediately correct them with implants 118, 120, and 122 at the time of surgery. As disclosed herein, the inventive method includes determining that a patient is a candidate for orthognathic surgery with effective evaluation, including 3D CT. CAD is used with CT to plan skeletal movements, and CAM devices are used to facilitate the operative procedure. Computer-simulated rearrangement of the skeleton is used to predict skeletal irregularities and imbalances that will remain after the skeletal rearrangement. Based on the information regarding skeletal irregularities and imbalances, CAD is used to plan implants to correct irregularities or imbalances.

Also according to the invention, the positions of the osteotomies for skeletal rearrangement are planned pre-operatively on computer, and cutting guides are designed on computer pre-operatively to guide the exact position and obliquity of the osteotomies. Fixation plates are made, potentially again by CAD/CAM based on electronic imaging, to maintain a new skeletal arrangement. With CAM and CAD used in coordination, a skeletal rearrangement procedure and an implant correction procedure can then be performed during the same operative procedure. In certain practices of the invention, implants 118, 120, and 122, fixation plates 105, and potentially other components for practicing the inventive method can be formed by three-dimensional printing using dedicated software while CAM of implants 118, 120, and 122 to correct irregularities or imbalances is performed using CAD. The position of osteotomies can thus be planned on the computer pre-operatively with cutting guides as in FIG. 4B in relation to a fibula and for use in relation to other skeletal locations designed to guide the exact position and obliquity of the osteotomies, and custom plates 105 and fasteners 107 as in FIG. 3 can be made to maintain the new skeletal arrangement.

Aspects of the present invention for one-stage CAD/CAM facial skeletal rearrangement and refinement, such as three-dimensional (3D) computerized tomographic (CT) imaging, three-dimensional printing, three-dimensional computer modeling and simulation, and other aspects exploited to permit skeletal rearrangement and refinement to be performed in a single operative procedure, can be informed by developments of the prior art. For instance, in certain practices of the invention, computer software sold under the registered trademark MATERIALISE by Materialise N.V. of Belgium can be used for computer-aided design and automated additive computer-aided manufacturing, commonly referred to as three-dimensional printing. Also to that end, the disclosures of U.S. Patent Application Publication No. 20060094951 and U.S. Pat. No. 7,747,305 to Dean et al., U.S. Patent Application Publication No. 2009/149977 of Schendell, U.S. Patent Application Publication No. 2017/0000564 of Gordon, and U.S. Pat. No. 10,595,942 to Rueber et al. are incorporated by reference as if fully set forth herein.

With certain details and embodiments of the present invention for one-stage CAD/CAM facial skeletal rearrangement and refinement disclosed, it will be appreciated by one skilled in the art that changes and additions could be made thereto without deviating from the spirit or scope of the invention. This is particularly true when one bears in mind that the presently preferred embodiments merely exemplify the broader invention revealed herein. Accordingly, it will be clear that those with certain major features of the invention in mind could craft embodiments that incorporate those major features while not incorporating all of the features included in the preferred embodiments.

Therefore, the following claims shall define the scope of protection to be afforded to the inventor. Those claims shall be deemed to include equivalent constructions insofar as they do not depart from the spirit and the scope of the invention. It must be further noted that a plurality of the following claims may express, or be interpreted to express, certain elements as means for performing a specific function, at times without the recital of structure or material. As the law demands, any such claim shall be construed to cover not only the corresponding structure and material expressly described in this specification but also all legally cognizable equivalents thereof.

I claim as deserving the protection of Letters Patent:

1. A method for corrective surgery with a rearrangement of skeletal features of a patient and a refinement of irregularities by alloplastic implant in a single surgery, the method comprising:
determining whether the patient is a candidate for corrective surgery;
pre-operatively imaging skeletal features to be rearranged to produce three-dimensional computer renderings of the skeletal features;
pre-operatively planning the rearrangement of skeletal features with computer-aided design;
pre-operatively predicting a skeletal irregularity to remain after the rearrangement of skeletal features with computer-aided design; and
pre-operatively designing an alloplastic implant based on the skeletal irregularity predicted to remain after the rearrangement of skeletal features with computer-aided design.

2. The method for corrective surgery of claim 1 further comprising manufacturing the alloplastic implant with computer-aided manufacture based on the computer-aided design.

3. The method for corrective surgery of claim 2 wherein the alloplastic implant is manufactured pre-operatively.

4. The method for corrective surgery of claim 2 wherein the alloplastic implant is manufactured in an additive manufacturing process.

5. The method for corrective surgery of claim 2 further comprising, in a single surgery, rearranging skeletal features in accordance with the pre-operative planning of the rearrangement of skeletal features and applying the alloplastic implant to the skeletal features in correction of the predicted skeletal irregularity.

6. The method for corrective surgery of claim 5 wherein the rearranging of skeletal features is performed under computerized tomographic imaging guidance.

7. The method for corrective surgery of claim 1 wherein the skeletal features comprise facial skeletal features.

8. The method for corrective surgery of claim 1 wherein the step of pre-operatively planning the rearrangement of skeletal features with computer-aided design includes planning by computer of osteotomies.

9. The method for corrective surgery of claim 8 further comprising designing cutting guides for the osteotomies by computer-aided design.

10. The method for corrective surgery of claim 1 wherein the step of determining whether the patient is a candidate for corrective surgery includes evaluation of the skeletal features by three-dimensional computerized tomographic imaging.

11. The method for corrective surgery of claim 1 wherein the pre-operative imaging comprises three-dimensional computerized tomographic imaging.

12. The method for corrective surgery of claim 1 further comprising creating a three-dimensional computer file of the alloplastic implant and manufacturing the alloplastic implant with computer-aided manufacture based on the computer-aided design and the three-dimensional computer file.

13. A method for corrective surgery with a rearrangement of skeletal features of a patient and a refinement of irregularities by alloplastic implant in a single surgery, the method comprising:
determining whether the patient is a candidate for corrective surgery;
pre-operatively imaging skeletal features to be rearranged to produce three-dimensional computer renderings of the skeletal features;
pre-operatively planning the rearrangement of skeletal features with computer-aided design;
pre-operatively predicting a skeletal irregularity to remain after the rearrangement of skeletal features with computer-aided design; and
pre-operatively designing an alloplastic implant based on the skeletal irregularity predicted to remain after the rearrangement of skeletal features with computer-aided design;
pre-operatively manufacturing the alloplastic implant with computer-aided manufacture based on the computer-aided design; and
rearranging skeletal features in accordance with the pre-operative planning of the rearrangement of skeletal features and applying the alloplastic implant to the skeletal features in correction of the skeletal irregularity predicted to remain after the rearrangement of skeletal features in a single surgery.

14. The method for corrective surgery of claim 13 wherein the alloplastic implant is manufactured in an additive manufacturing process.

15. The method for corrective surgery of claim 13 wherein the rearranging of skeletal features is performed under computerized tomographic imaging guidance.

16. The method for corrective surgery of claim 13 wherein the skeletal features comprise facial skeletal features.

17. The method for corrective surgery of claim 13 wherein the step of pre-operatively planning the rearrangement of skeletal features with computer-aided design includes planning by computer of osteotomies.

18. The method for corrective surgery of claim 17 further comprising designing cutting guides for the osteotomies by computer-aided design.

19. The method for corrective surgery of claim 13 wherein the step of determining whether the patient is a candidate for corrective surgery includes evaluation of the skeletal features by three-dimensional computerized tomographic imaging.

20. The method for corrective surgery of claim 13 wherein the pre-operative imaging comprises three-dimensional computerized tomographic imaging.
